(19) United States
(12) Patent Application Publication
(10) Pub. No.: US 2007/0276163 A1
(43) Pub. Date: Nov. 29, 2007

(54) Processes for Preparing N-Acetylcolchinol & Intermediates Used in Such Processes

(76) Inventors: Matthew Evans, Cheshire (GB); John Leonard, Cheshire (GB); Timothy J. Lilley, Cheshire (GB); John Whittall, Cheshire (GB)

Correspondence Address: LADAS & PARRY, 26 WEST 61ST STREET, NEW YORK, NY, 10023, US

(21) Appl. No.: 10/584,175

(22) PCT Filed: Dec. 21, 2004
(86) PCT No.: PCT/GB04/05389

§ 371 Date: Apr. 4, 2007

(30) Foreign Application Priority Data
Dec. 23, 2003 (GB) 0329771.0

(51) Int. Cl. C07C 205/13; C07C 409/16

(52) U.S. Cl. 568/558; 568/568; 568/707

(57) ABSTRACT

A process for the preparation of ZD6126 Phenol: 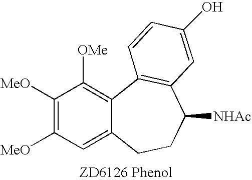
from allocolchicine or an ester derivative thereof of formula (I), or from a ZD6126 Alcohol of the Formula (II): 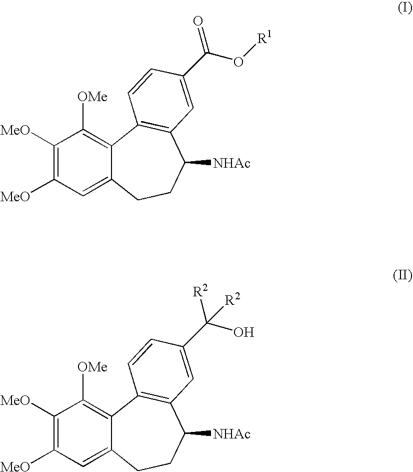
wherein R¹ and R² are as defined in the description. Also claimed are intermediates, processes for their preparation and the use of the intermediates in the manufacture of ZD6126 Phenol.

The present invention relates to processes for synthesising N-((S)-3-hydroxy 9,10,11-trimethoxy-6,7-dihydro-5H-dibenzo[a,c]cyclohepten-5-yl)-acetamide (hereafter ZD6126 Phenol) from ZD6126 Alcohol or allocolchicine or an ester derivative thereof, to intermediates used in such processes, to processes for the manufacture of such intermediates and to the use of said intermediates in the manufacture of ZD6126 Phenol.

ZD6126 Phenol is also known as N-acetylcolchinol: 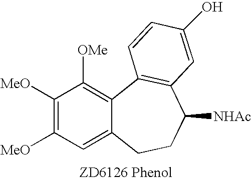
and is an intermediate useful in the synthesis of (5S)-5-(acetylamino)-9,10,11-trimethoxy-6,7-dihydro-5H-dibenzo[a,c]cyclohepten-3-yl dihydrogen phosphate or N-acetylcolchinol-O-phosphate (hereafter ZD6126): 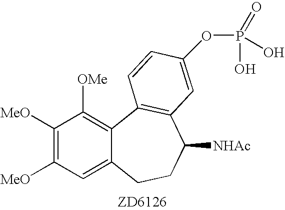
a potent vascular targeting agent.

ZD6126 is described in International Patent Application Publication No. WO 99/02166 (Example 1). It has been reported that ZD6126 selectively disrupts tumour vasculature leading to vessel occlusion and extensive tumour necrosis (Davis, P. D., Hill, S. A., Galbraith, S. M., et al., Proc. Am. Assoc. Cancer Res., 2000; 41: 329). ZD6126 is therefore useful in the treatment of cancer.

WO 99/02166 describes a synthesis of ZD6126 Phenol from colchicine which comprises (a) an acid hydrolysis using hydrochloric acid at a temperature of at or near 100° C., followed by (b) treatment of the resulting hydroxy ketone intermediate with alkaline hydrogen peroxide to give ZD6126 Phenol. This is illustrated in Scheme A.

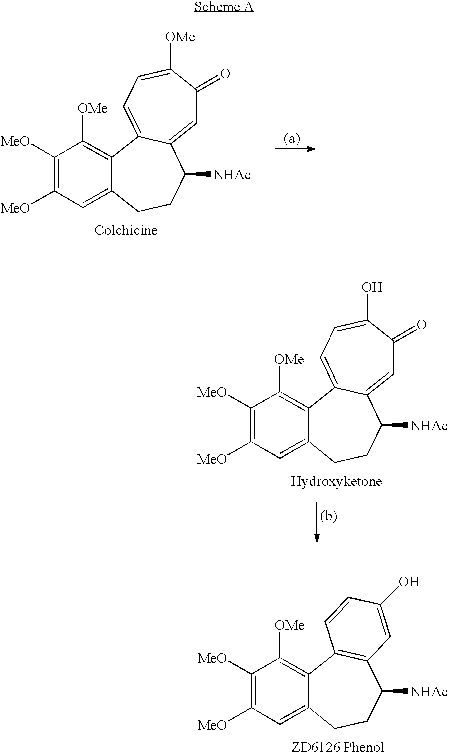

Santavy, F., in Collect. Czech. Chem. Commun., 1949, 14 532-535 reports yields for this synthesis of 79% for step (a) and 25% for step (b) leading to an overall yield of 19%. This is obviously a less than ideal synthesis for use on a large scale.

There is therefore a need for an alternative process for the preparation of ZD6126 Phenol.

Allocolchicine or ester derivatives thereof may be prepared from colchicine. For example allocolchicine itself can be prepared in 90% yield by treatment of colchicine with sodium methoxide in methanol (Fernholz, V., Justus Liebigs Ann., 1950, 568, 63-72).

Boger et al (J. Org. Chem. 1986, 51, 5436-5439) describes the small-scale conversion of certain benzylic secondary or tertiary alcohols to the corresponding phenol.

The present invention relates to a novel process for the synthesis of ZD6126 Phenol from allocolchicine or an ester derivative thereof via an alcohol (ZD6126 Alcohol defined herein) which gives a surprisingly high yield of 75% (67% from colchicine).

According to a first aspect of the present invention there is provided a process for the preparation of ZD6126 Phenol from a ZD6126 Alcohol of formula (II): 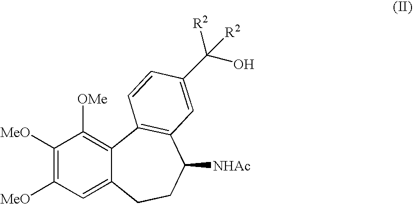
wherein R² are each independently hydrogen, C₁₋₄alkyl or aryl which comprises: reacting said ZD6126 Alcohol of formula (II) with an acid catalyst and an oxidising agent.

Particular values for R² are C₁₋₄alkyl. More particular values for R² are hydrogen, methyl, ethyl, butyl, t-butyl and phenyl. In one aspect of the invention both R² are methyl. In another aspect of the invention one or both of the groups R² can be hydrogen.

Particular oxidising agents for use in the reactions described herein are peroxides, hydroperoxides or peroxyacids. More particularly the oxidising agent is hydrogen peroxide, which is conveniently used as an aqueous solution, for example a solution containing from 10 to 60% (w/v) peroxide. In an embodiment, a molar excess of oxidising agent relative to the ZD6126 Alcohol is used, for example a molar excess of approximately 3 or more.

A range of acids haven been shown to be effective acid catalysts for use in the reaction. Particular acid catalysts for use in the reactions described herein include, for example inorganic acids such as sulphuric acid and organic acids such as carboxylic and sulfonic acids. Particular organic carboxylic and sulfonic acids include, for example aryl or aliphatic carboxylic or sulfonic acids. Suitable aryl carboxylic or sulfonic acids include for example benzene substituted by one or more carboxylic or sulfonic acid group, and wherein the benzene is optionally further substituted by for example one or more substituents selected from C₁₋₄-alkyl, hydroxy and halogeno. Suitable aliphatic carboxylic or sulfonic acids include for example a saturated or unsaturated aliphatic group such as a C₁₋₆alkane or C₂₋₆alkene which carries one of more carboxylic or sulfonic acid group, and wherein the aliphatic group is optionally further substituted by one or more substituents selected from for example halogeno and hydroxy. Particular acid catalysts include for example methanesulfonic acid, trifluoroacetic acid or toluenesulfonic acid. More particularly the acid catalyst is a sulfonic acid, such as a C₁₋₄alkanesulfonic acid or an aryl sulfonic acid, for example methanesulfonic acid or para-toluenesulfonic acid. A particular acid catalyst is methanesulfonic acid. Suitably the molar ratio of acid catalyst to ZD6126 Alcohol is approximately equimolar.

Examples of acid catalysts that have been evaluated are shown in Table 1.

TABLE 1

| Acid catalyst | Conversion to ZD6126 Phenol/HPLC area % |
|---|---|
| Trifluoroacetic acid | 51 |
| pTSA/toluene | 76 |
| pTSA/water^{[1]} | 94 |
| MeSO₃H^{[2]} | 94-96 |

wherein pTSA is para-toluenesulfonic acid.
Notes: [1] water present as water of hydration

[2] methane sulfonic acid was used as an aqueous solution containing up to 30% (w/v) water.

The reaction is conveniently carried out in the presence of a solvent. Suitable solvents for use in the reaction include, for example an aromatic solvent such as xylene, toluene, chlorobenzene or trifluorotoluene; an ester such as butyl acetate; an ether such as tetrahydrofuran or methyl tert-butyl ether; or a mixture of two or more of the solvents.

Examples of solvents that have been investigated for the conversion of a ZD6126 Alcohol of the formula (II) in which each R² is methyl to ZD6126 Phenol are shown in Table 2:

TABLE 2

| Solvent | % Conversion to ZD6126 Phenol |
|---|---|
| Butyl acetate | 74 |
| Toluene | 95 |
| Trifluorotoluene | 78 |
| Methyl t-butyl ether | 87 |
| Chlorobenzene | 91 |
| Xylene | 93 |

We have surprisingly found that despite the relatively low solubility of ZD6126 alcohol in aromatic solvents, such solvents provide a high yield of the ZD6126 Phenol. Accordingly in an embodiment the solvent is an aromatic solvent, for example toluene, trifluorotoluene, chlorobenzene or xylene, more particularly the solvent is toluene, chlorobenzene or xylene, still more particularly the solvent is toluene or chlorobenzene or a mixture thereof.

At the completion of the reaction the reaction may be quenched to remove excess oxidising agent by adding a suitable quenching agent such as sodium thiosulfate.

The reaction is suitably carried out at elevated temperature, for example from 30 to 70° C., such as about 50° C.

In a particular embodiment of this aspect of the invention each R² is C₁₋₄alkyl such as methyl; the acid catalyst is selected from methanesulfonic acid and para-toluenesulfonic acid (optionally in the presence of small quantities of water); the oxidising agent is as hereinbefore defined such as hydrogen peroxide; and wherein the reaction is carried out in a solvent as hereinbefore defined, particularly an aromatic solvent selected from toluene or chlorobenzene, or a mixture thereof.

Conveniently, transformation of ZD6126 Alcohol into ZD6126 Phenol is brought about by dual addition of an oxidizing agent, more particularly hydrogen peroxide, and an acid catalyst, more particularly methanesulfonic acid, at an elevated temperature, for example 50° C. By the term dual addition is meant the substantially simultaneous addition of the acid catalyst and oxidising agent to the reaction mixture containing the ZD6126 alcohol. Suitably the dual addition is carried out by adding the acid catalyst and oxidising agent as separate feeds to the ZD6126 Alcohol at about the same time. This means of dual addition avoids the need to prepare a pre-mix of acid and oxidising agent, which under certain circumstances, may be hazardous.

We have found that the ZD6126 Alcohol used as a starting material can be prepared from allocolchicine or an ester derivative thereof in high yield. The preparation of ZD6126 Phenol from allocolchicine or an ester derivative thereof forms a further aspect of the invention.

According to a second aspect of the present invention there is provided a process for the preparation of ZD6126 Phenol from an allocolchicine or an ester derivative thereof of formula (I): 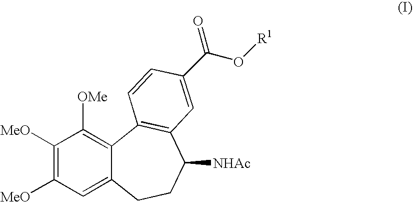
wherein R¹ is hydrogen, C₁₋₆alkyl or aryl; which comprises:
a) reacting said allocolchicine or an ester derivative thereof of formula (I) with a suitable organometallic reagent and/or a suitable reducing agent; in one or more ethereal solvents to form ZD6126 Alcohol of formula (II): 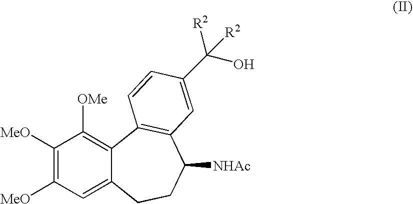
   wherein R² are each independently hydrogen, C₁₋₄alkyl or aryl; and
b) reacting ZD6126 Alcohol of formula (II) with an acid catalyst and an oxidising agent.

For the avoidance of doubt, the phrase “suitable organometallic reagent and/or a suitable reducing agent” may be selected such that the two R² groups introduced are the same or different.

In this specification, the term “aryl” refers to a 4-10 membered aromatic mono or bicyclic ring containing 0 to 5 heteroatoms independently selected from nitrogen, oxygen or sulphur wherein said aryl may be optionally substituted. Suitable optional substituents for “aryl” include halo, C₁₋₆alkyl, C₁₋₆alkoxy. Examples of “aryl” include phenyl; phenyl substituted by halo, C₁₋₆alkyl or C₁₋₆alkoxy; and certain herteroaromatics, for example pyridyl. In particular “aryl” refers to phenyl.

In this specification the term “alkyl” includes both straight and branched chain alkyl groups but references to individual alkyl groups such as “propyl” are specific for the straight chain version only. For example, “C₁₋₆alkyl” and “C₁₋₄alkyl” includes propyl, isopropyl and t-butyl. However, references to individual alkyl groups such as ‘propyl’ are specific for the straight chained version only and references to individual branched chain alkyl groups such as ‘isopropyl’ are specific for the branched chain version only. Examples of “C₁₋₆alkyl” and “C₁₋₄alkyl” include methyl, ethyl, propyl, isopropyl and t-butyl. The term “halo” refers to fluoro, chloro, bromo and iodo. Examples of “C₁₋₆alkoxy” include methoxy, ethoxy and propoxy.

Particularly, in the formation of ZD6126 Alcohol from allocolchicine or an ester derivative thereof, the allocolchicine or ester derivative thereof is added to >3 mole equivalents of the suitable organometallic reagent and/or suitable reducing agent, preferably maintaining the reaction temperature below ambient.

In a compound of formula (I) when R¹ is methyl this is allocolchicine. In an embodiment R¹ is C₁₋₆alkyl or aryl. Suitably R¹ is C₁₋₄alkyl. In another aspect R¹ is methyl or ethyl. In a further aspect R¹ is methyl.

Particular values for R² are as hereinbefore defined such as C₁₋₄alkyl. More particular values for R² are hydrogen, methyl, ethyl, butyl, t-butyl and phenyl. In one aspect of the invention both R² are methyl. In another aspect of the invention one or both of the groups R² can be hydrogen.

Suitable organometallic reagents are those that introduce an R² group that is C₁₋₄alkyl or aryl. Examples of suitable organometallic reagents for use in the reactions described herein include compounds of the formula R²—X, wherein R² is as hereinbefore defined and X is lithium or a magnesium halide such as magnesium chloride, bromide or iodide. Particular organometallic reagents include for example, methyllithium, ethyllithium, methylmagnesium chloride, methylmagnesium bromide, ethylmagnesium chloride, ethylmagnesium bromide, butyllithium and phenyllithium. More particularly the organometallic reagent is selected from methyllithium or ethyllithium. Still more particularly the organometallic reagent is methyllithium.

Suitable reducing agents are those that introduce an R² group that is hydrogen. Examples of suitable reducing agents for use in the reactions described herein include, for example lithium aluminium hydride, di-isobutyl aluminium hydride, sodium borohydride or a borane reducing agent, for example a borane-tetrahydrofuran or borane-dimethylsulfide complex.

In one aspect of the invention one or more suitable organometallic reagents are used in step a). This results in a tertiary ZD6126 Alcohol.

In another aspect of the invention a suitable organometallic reagent and a suitable reducing agent are used in step a). In the first instance allocolchicine or an ester derivative thereof of formula (I) wherein R¹ is C₁₋₆alkyl or aryl is converted into a ketone by reaction with one equivalent of a suitable organometallic reagent, for example methyllithium, ethyllithium, methylmagnesium chloride, methylmagnesium bromide, ethylmagnesium chloride, ethylmagnesium bromide, butyllithium or phenyllithium. The ketone is then converted to ZD6126 Alcohol by reaction with a suitable reducing agent such as lithium aluminium hydride, di-isobutyl aluminium hydride or sodium borohydride. This results in a secondary ZD6126 Alcohol.

In a further aspect of the invention one or more suitable reducing reagents are used in step a). This results in primary ZD6126 Alcohol.

The skilled person will appreciate that when R¹ is hydrogen, the compound of formula (I) is reacted with a reducing agent to give a primary ZD6126 Alcohol. Accordingly when the allocolchicine or an ester thereof of formula (I) is reacted with an organometallic reagent alone R¹ is C₁₋₆alkyl or aryl.

In another aspect of the invention step a) might be conducted in the presence of an alkali metal halide. We have found that the use of an alkali metal halide can improve the yield of the ZD6126 alcohol. Particular alkali metal halides are lithium chloride or lithium bromide. A more particular alkali metal halide is lithium bromide.

Particular ethereal solvents for use in the reactions described herein are tetrahydrofuran, diethyl ether, diethoxymethane, 2-ethoxyethylether, 2-methoxyethyl ether and dimethoxy ethane or a mixture of one or more of these solvents. Yields for step a) conducted in various ethereal solvents are given in Table 3. Conveniently, the ethereal solvent used in the reactions described herein is a mixture of tetrahydrofuran and diethoxymethane. In another aspect, more particularly the ethereal solvent used in the reactions described herein is diethyl ether. In another aspect, more particularly the ethereal solvent used in the reactions described herein is 2-ethoxyethylether. In another aspect, more particularly the ethereal solvent used in the reactions described herein is 2-methoxyethyl ether. In another aspect, more particularly the ethereal solvent used in the reactions described herein is dimethoxy ethane. In another aspect, more particularly the ethereal solvent used in the reactions described herein is tetrahydrofuran.

TABLE 3

| Solvent | % Conversion to Alcohol |
|---|---|
| Tetrahydrofuran | 91 |
| Diethyl ether | 90 |
| Dimethoxy ethane | 65 |
| 2-Ethoxyethyl ether | 65 |
| 2-Methoxyethyl ether | 80 |

Suitably the reaction is carried out at a temperature below ambient, for example below 20° C., particularly at 0° C. or less, for example at less than −5° C.

In a particular embodiment, the allocolchicine or ester derivative thereof of formula (I) is added to a reaction vessel containing the organometallic regent. Suitably the allocolchicine is added to a reaction mixture containing the organometallic reagent and the ethereal solvent. The reaction mixture may be agitated, for example by stirring, during the addition of the organometallic reagent and subsequent reaction. Conveniently the allocolchicine or ester derivative thereof of formula (I) is added to the organometallic reagent as a solution or slurry in a suitable solvent, for example an ethereal solvent such as tetrahydrofuran. We have surprisingly found that the addition of the allocolchicine to the organometallic reagent significantly reduces the formation of undesirable ketone by-products compared to adding the organometallic to the allocolchicine. The reduced by-product formation is particularly marked when the organometallic reagent is methyllithium.

Step b) of the process is an acid catalysed oxidative rearrangement to form ZD6126 Phenol plus a carbonyl compound as described in relation to the first aspect of the invention. Particular oxidising agents and acid catalysts are as hereinbefore described in relation to the first aspect of the invention, for example hydrogen peroxide and methanesulfonic acid.

Suitably the reaction is carried out in the presence of a solvent as hereinbefore described in relation to the first aspect of the invention, such as toluene, xylene, chlorobenzene, trifluorotoluene, methyl tert-butyl ether, butyl acetate or tetrahydrofuran and particularly an aromatic solvent such as toluene, xylene, chlorobenzene or trifluorotoluene, more particularly chlorobenzene or toluene, or a mixture thereof.

The conversion of allocolchicine or ester derivative thereof of formula (I) to ZD6126 Phenol may be effected in one stage, without isolating the ZD6126 alcohol following step a). Alternatively the process according to the second aspect of the invention may be carried out in two consecutive stages wherein the ZD6126 alcohol is isolated prior to conversion to the ZD6126 Phenol in step b) of the process.

Conveniently, the ethereal solution of ZD6126 Alcohol, as prepared in Step a) is converted into a solution in toluene (or other suitable solvent) by azeotropic distillation. Direct transformation of ZD6126 Alcohol into ZD6126 Phenol is then brought about by addition of an oxidizing agent, more particularly hydrogen peroxide, and an acid catalyst, more particularly methanesulfonic acid, at an elevated temperature, for example 50° C. as described hereinbefore in relation to the first aspect of the invention. Suitably the acid and oxidizing agent are added to the ZD6126 Alcohol by means of a dual addition procedure of the acid and oxidizing agent as described hereinbefore.

In a particular embodiment of this aspect of the invention there is provided a process for the preparation of ZD6126 Phenol comprising:

a) reacting said allocolchicine or an ester derivative thereof of formula (I) as herein before defined wherein R¹ is C₁₋₄alkyl (particularly methyl) with a suitable organometallic reagent selected from methyllithium, methylmagnesium chloride, methylmagnesium bromide, ethylmagnesium chloride, ethylmagnesium bromide, butyllithium and phenyllithium (particularly methyllithium);

in one or more ethereal solvents selected from tetrahydrofuran, diethyl ether, diethoxymethane, 2-ethoxyethylether, 2-methoxyethyl ether and dimethoxy ethane or a mixture of one or more of these solvents (particularly a solvent selected from tetrahydrofuran and diethoxymethane or a mixture thereof);

to form ZD6126 Alcohol of formula (II) as hereinbefore defined wherein each R² is C₁₋₄alkyl (particularly methyl); and

b) reacting said ZD6126 Alcohol of formula (II) with an acid catalyst (particularly methanesulfonic acid) and an oxidising agent (particularly hydrogen peroxide); and wherein step b) is carried out in an aromatic solvent selected from toluene, chlorobenzene and xylene (particularly the solvent is toluene or chlorobenzene, or a mixture thereof). Suitably in step (a) the organometallic reagent is methyllithium and the allocolchicine or an ester derivative thereof of formula (I) is added to a reaction mixture comprising the methyllithium. Suitably in step (b) of the process, the acid and oxidizing agent are added to the ZD6126 Alcohol by means of a dual addition procedure of the acid and oxidizing agent as described hereinbefore.

In a further aspect of the invention, ZD6126 Alkene, ZD6126 Hydroperoxide and ZD6126 Reactive Dimer are known by-products (and possible intermediates) of the reaction. The present inventions have demonstrated that each of these compounds can be converted into ZD6126 Phenol. These compounds are thus provided as a further feature of the invention.

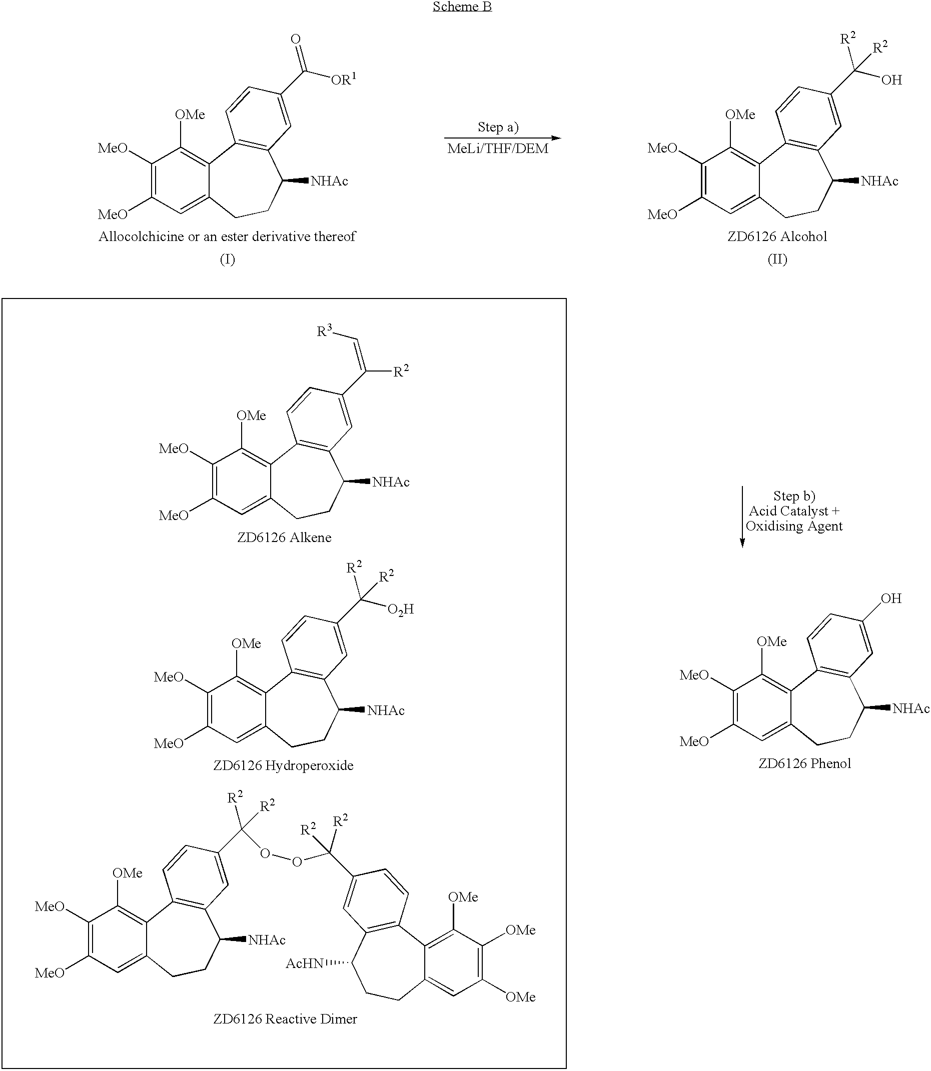

R³ is hydrogen or C₁₋₃alkyl and R³ is always one carbon shorter than the C₁₋₄alkyl R² group that formed it. For example if said R² was methyl, R³ is hydrogen. If R² was ethyl, R³ is methyl. If R² was propyl, R³ is ethyl and so on.

The skilled person will appreciate that ZD6126 Alkene will not be formed unless at least one R² in the ZD6126 Alcohol is C₁₋₄alkyl. However, conversion of ZD6126 Alcohol to ZD6126 Phenol will occur even if neither R² is C₁₋₄alkyl.

As mentioned hereinbefore, in one aspect of the invention, the conversion of allocolchicine or an ester derivative thereof into ZD6126 Phenol may be effected in one stage, without isolation of ZD6126 Alcohol. This has the advantage that it allows the steps a) and b) of the process to be carried out in a single reaction vessel. In another aspect of the invention allocolchicine or an ester derivative thereof is converted into ZD6126 Alcohol, which is isolated as a solid following step a). In a further aspect of the invention ZD6126 Alcohol is converted into ZD6126 Phenol in a single stage.

In another aspect of the invention ZD6126 Alcohol is converted into ZD6126 Hydroperoxide, which is isolated. In a further aspect of the invention ZD6126 Hydroperoxide is converted into ZD6126 Phenol.

In another aspect of the invention ZD6126 Alcohol is converted into ZD6126 Alkene, which is isolated. In a further aspect of the invention ZD6126 Alkene is converted into ZD6126 Phenol.

In another aspect of the invention ZD6126 Alcohol is converted into ZD6126 Reactive Dimer which is isolated. In a further aspect of the invention ZD6126 Reactive Dimer is converted into ZD6126 Phenol.

Certain intermediates described herein are novel and are provided as another aspect of the present invention.

According to another aspect of the present invention there is provided ZD6126 Alcohol of formula (II) (as depicted above) with the proviso that R² cannot both be methyl or both be hydrogen.

According to another aspect of the present invention there is provided a process for the preparation of a ZD6126 Alcohol of the formula (II) wherein R² are each independently hydrogen, C₁₋₄alkyl or aryl which comprises reacting a compound of formula (I) (as depicted above—allocolchicine or an ester derivative thereof) with a suitable organometallic reagent and/or suitable reducing agent in one or more ethereal solvents. Suitable reagents, solvents and conditions for this reaction are as described herein in relation to step (a) of the process according to the second aspect of the invention.

According to another aspect of the present invention there is provided the use of a ZD6126 Alcohol of formula (II) in a process for the preparation of ZD6126 Phenol.

According to another aspect of the present invention there is provided ZD6126 Alkene of formula (III): 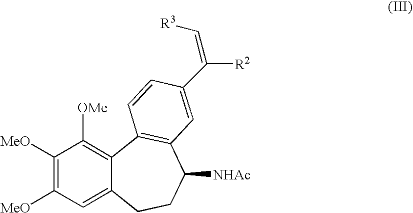
wherein R² is hydrogen, C₁₋₄alkyl or aryl and R³ is hydrogen or C₁₋₃alkyl.

According to another aspect of the present invention there is provided a process for the preparation of ZD6126 Alkene of formula (III) (as depicted above) which comprises reacting a ZD6126 Alcohol of the formula (II) wherein at least one R² group is C₁₋₄alkyl with an acid catalyst. Suitable acid catalysts are as hereinbefore defined in relation to the first aspect of the invention, for example methanesulfonic acid. The reaction is conveniently carried out in the presence of a suitable solvent, for example an ether such as tetrahydrofuran. The reaction is suitably carried out at elevated temperature, for example from 30 to 70° C., for example about 60° C.

According to another aspect of the present invention there is provided the use of ZD6126 Alkene of formula (III) in a process for the preparation of ZD6126 Phenol.

According to another aspect of the present invention there is provided a process for the preparation of ZD6126 Phenol which comprises reacting a ZD6126 Alkene of formula (III) (as depicted above) with an acid catalyst and an oxidising agent.

Suitable acid catalysts and oxidising agents for use in this reaction are as hereinbefore defined in relation to the first aspect of the invention. For example, a suitable acid catalyst includes methanesulfonic acid, trifluoroacetic acid or toluenesulfonic acid. A particular acid catalyst is methanesulfonic acid. An example of a suitable oxidising agent includes a peroxide, particularly hydrogen peroxide. The reaction is conveniently carried out in a suitable solvent, for example an aromatic solvent such as chlorobenzene or toluene, or a mixture thereof. Suitably the reaction is carried out at elevated temperature, for example from 30 to 70° C., for example about 50° C.

According to another aspect of the present invention there is provided ZD6126 Hydroperoxide of formula (IV): 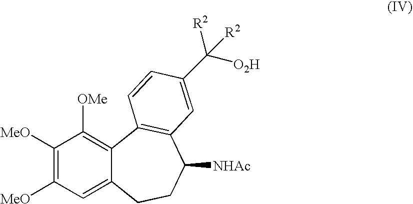
wherein R² are each independently hydrogen, C₁₋₄alkyl or aryl.

According to another aspect of the present invention there is provided a process for the preparation of ZD6126 Hydroperoxide of formula (IV) (as depicted above) which comprises reacting a ZD6126 Alcohol of the formula (II) with an acid catalyst and oxidising agent conveniently in the presence of a solvent. Suitable acid catalysts and oxidising agents for use in this reaction are as hereinbefore defined in relation to the first aspect of the invention. For example, a suitable acid catalyst includes methanesulfonic acid. An example of a suitable oxidising agent includes a peroxide, particularly hydrogen peroxide. A suitable solvent is for example an ester such as butyl acetate, or particularly a mixture of an ester and water such as butyl acetate and water. Suitably the reaction is carried out at a temperature of 30° C. or below because this favours formation of the ZD6126 Hydroperoxide over the ZD6126 Phenol.

According to another aspect of the present invention there is provided a process for the preparation of ZD6126 Hydroperoxide of formula (IV) (as depicted above) wherein at least one R² group is C₁₋₄alkyl which comprises reacting a ZD6126 Alkene of formula (III) with an oxidising agent, conveniently in the presence of a solvent. Suitable oxidising agents are as hereinbefore defined in relation to the first aspect of the invention, for example a peroxide such as hydrogen peroxide. Suitable solvents for use in this reaction include, for example an aromatic solvent as hereinbefore defined such as toluene or chlorobenzene, or a mixture thereof.

According to another aspect of the present invention there is provided the use of ZD6126 Hydroperoxide of formula (IV) in a process for the preparation of ZD6126 Phenol.

According to another aspect of the present invention there is provided a process for the preparation of ZD6126 Phenol which comprises reacting a ZD6126 Hydroperoxide of formula (IV) (as depicted above) with an acid catalyst. Suitable acid catalysts are as defined hereinbefore in relation to the first aspect of the invention, for example methanesulfonic acid. The reaction is conveniently carried out in the presence of a suitable solvent, for example an aromatic solvent as hereinbefore defined such as toluene or chlorobenzene, or a mixture thereof. Suitably the reaction is carried out at elevated temperature, for example from 30 to 70° C., for example about 50° C.

According to another aspect of the present invention there is provided ZD6126 Reactive Dimer of formula (V): 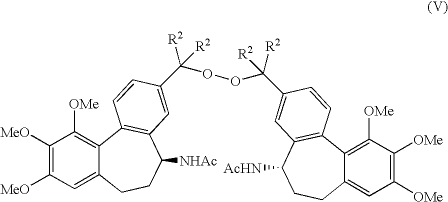
wherein R² are each independently hydrogen, C₁₋₄alkyl or aryl.

According to another aspect of the present invention there is provided a process for the preparation of ZD6126 Reactive Dimer of formula (V) (as depicted above) which comprises reacting ZD6126 Alcohol with an oxidizing agent and an acid catalyst.

Suitable oxidising agents are as hereinbefore defined in relation to the first aspect of the invention such as hydrogen peroxide. Suitable acid catalysts are as hereinbefore defined in relation to the first aspect of the invention, for example methane sulfonic acid. The reaction is conveniently carried out in the presence of a suitable solvent, for example an aromatic solvent such as toluene or chlorobenzene, or a mixture thereof. Suitably the reaction is carried out at elevated temperature, for example from 30 to 70° C., for example about 40° C. In an embodiment, the reaction is quenched shortly after adding the oxidising agent and acid catalyst to the ZD6126 alcohol, for example within 10 minutes, suitably less than 5 minutes after adding the acid and oxidising agent. Suitable quenching agents are well known, for example when the oxidising agent is hydrogen peroxide sodium thio sulfate may be used.

According to another aspect of the present invention there is provided the use of ZD6126 Reactive Dimer in a process for the preparation of ZD6126 Phenol.

According to another aspect of the present invention there is provided a process for the preparation of ZD6126 Phenol which comprises reacting a ZD6126 Reactive Dimer of formula (V) (as depicted above) with an acid catalyst and oxidising agent. Suitable acid catalysts and oxidising agents for use in this reaction are as hereinbefore defined in relation to the first aspect of the invention. For example, a suitable acid catalyst includes methanesulfonic acid. An example of a suitable oxidising agent includes a peroxide, particularly hydrogen peroxide. The reaction is conveniently carries out in the presence of a solvent, for example an aromatic solvent as hereinbefore defined such as toluene or chlorobenzene, or a mixture thereof. Suitably the reaction is carried out at a temperature of from 30 to 70° C., for example about 50° C.

The products of the reactions described herein may be isolated using conventional methods well known in the art and as illustrated in the Examples herein.

### EXAMPLES

The invention will now be illustrated in the following non limiting examples, in which standard techniques known to the skilled chemist and techniques analogous to those described in these examples may be used where appropriate, and in which, unless otherwise stated:
(i) evaporations were carried out by rotary evaporation in vacuo and work up procedures were carried out after removal of residual solids such as drying agents by filtration;
(ii) all reactions were carried out under an inert atmosphere at ambient temperature, typically in the range 18-25° C., with solvents technical grade under anhydrous conditions, unless otherwise stated;
(iii) the structures of the end products of the formula (I) were generally confirmed by nuclear (generally proton) magnetic resonance (NMR) and mass spectral techniques; magnetic resonance chemical shift values were measured in deuterated dimethyl sulphoxide (unless otherwise stated) on the delta scale (ppm downfield from tetramethylsilane); proton data is quoted unless otherwise stated; spectra were recorded on a on a Bruker DRX500 spectrometer; and peak multiplicities are shown as follows: s, singlet; d, doublet; dd, double doublet; t, triplet; tt, triple triplet; q, quartet; tq, triple quartet; m, multiplet; br, broad; LCMS were recorded on a Waters ZQ Mass Spec Detector, LC column was a SB C8 150 mm×3.0 mm 3.5 um (Agilent Zorbax), detection with a HP1100 with a Diode Array Detector; unless otherwise stated the mass ion quoted is [M+H]⁺;
(iv) the following abbreviations may be used hereinbefore or hereinafter:
   THF tetrahydrofuran;
   BuOAc butyl acetate; and
   eq. equivalent; and
(v) the term Rel. Vols (or Vols) refers to the relative amount of solvent used in millilitres, relative to the amount of the main reaction substrate in grams.

### Example 1

### Allocolchicine to ZD6126 Alcohol (wherein R² are both methyl in formula (II))

To a stirred solution of methyllithium (4 mole equivalents of a 3 M solution) in diethoxymethane and THF (3 Rel. Vols), at <−5° C., was added a slurry of allocolchicine in THF (3-7 Rel. Vols), over 1 hour. After a further 1 hour (or when no allocolchicine remained by HPLC) the mixture was treated, first with aqueous THF (3 mole equiv. water made-up to 1 Rel Vol with THF), then with water (4 Rel. Vols). Toluene (15 Rel. Vols) was then added and the aqueous layer was removed. The mixture was washed further with water (3×2 Rel. Vols). mixture and was then distilled under reduced pressure to a volume of 5 Rel. Vols. A further charge of toluene (20 Rel. Vols) was added to the mixture and it was further distilled under reduced pressure to a volume of about 10 Rel. Vols. The mixture was then cooled and the solid was filtered off, washed with toluene (2 Rel. Vols) and then dried in a vacuum oven at 50° C. The isolated yield of ZD6126 Alcohol was 85%: MS, 382 [M−OH]⁺-(100%); δ^{H} ppm (500 MHz, DMSO-D₆) 1.46 (3 H, s, CHCH₃), 1.49 (3 H, s, CHCH₃), 1.89 (3 H, s, COCH₃), 1.89 (1 H, m, CH₂CH₂), 2.04 (1 H, m, CH₂CH₂), 2.15 (1 H, m, CH₂CH₂), 2.47 (1 H, m, CH₂CH₂), 3.51 (3 H, s, OCH₃), 3.78 (3 H, s, OCH₃), 3.83 (3 H, s, OCH₃), 4.59 (1 H, m, CH₂CH—NH), 6.77 (1 H, s, Ar—H), 7.24 (1 H, d, J8, Ar—H), 7.37 (1 H, dd, J8, 2, Ar—H), 7.57 (1H, d, J2, Ar—H), 8.45 (1 H, d, J8.5, NH).

### Example 2

### Allocolchicine to ZD6126 Phenol

To a stirred solution of methyllithium (4 mole equivalents of a 3 M solution) in diethoxymethane and THF (3 Rel. Vols), at <−5° C., was added a slurry of allocolchicine in THF (3-7 Rel. Vols), over 1 hour. After a further 1 hour (or when no allocolchicine remained by HPLC) the mixture was treated, first with aqueous THF (3 mole equiv. water made-up to 1 Rel Vol with THF), then with water (4 Rel. Vols). Toluene (15 Rel. Vols) was then added and the aqueous layer was removed. The mixture was washed further with water (3×2 Rel. Vols). mixture and was then distilled under reduced pressure to a volume of 5 Rel. Vols. A further charge of toluene (20 Rel. Vols) was then added to the mixture and it was further distilled under reduced pressure to a volume of approximately 18 Rel. Vols.

To the mixture from above, at 50° C., with stirring was added simultaneously, methane sulfonic acid (1 mol. eq.) and hydrogen peroxide (3 mol. eq.) over 1 hour. Following a further 1 hour, the mixture was quenched by the addition of sodium thiosulfate solution (1 M, 3 mol. eq.) and cooled to 20° C. Potassium hydroxide (49% (w/v), 7 mol eq.) was added and the layers were separated, retaining the lower aqueous layer. To this solution was added water (1.7 vols) and BuOAc (17 vols) and the pH was adjusted to 7 by the addition of hydrochloric acid (2.5 M). The layers were again separated, this time retaining the upper organic layer, which was washed with water wash (4.25 vols). The volume of the BuOAc solution was then reduced to approximately 8.5 Rel. Vols. by distillation under reduced pressure. Heptane (8.5 Rel. vols) was added at approximately 80° C. and the mixture was cooled to 0° C. over 4 hours. The solid was filtered off, washed with a mixture of heptane and BuOAc (1.7 Rel. vols of each) then with heptane (3.4 vols) and finally dried in vacuum oven at 50° C. Overall isolated yield of ZD6126 Phenol, form allocolchicine was approximately 75%. Data for ZD6126 Phenol: MS 358 [M+H]⁺ (75%), 299 [M−NHCOMe] (100%); δ^{H} ppm (500 MHz, DMSO-D₆) 1.82-1.90 (1 H, m, CH₂CH₂), 1.88 (3 H, s, COCH₃), 2.04-2.17 (2 H, m, CH₂CH₂), 2.47 (1 H, dd, J11.5, 5, CH₂CH₂), 3.46 (3 H, s, OCH₃), 3.77 (3 H, s, OCH₃), 3.82 (3 H, s, OCH₃), 4.44-4.50 (1 H, m, CH₂CH—NH), 6.69 (1 H, dd, J8.5, 2, Ar—H), 6.74 (1 H, s, Ar—H), 6.77 (1 H, d, J2.5), 7.12 (1 H, d, J8.5), 9.40 (1 H, s, OH).

### Example 3

### ZD6126 Alcohol (wherein R² are both methyl in formula (II)) to ZD6126 Phenol

To a stirred mixture of ZD6126 Alcohol in toluene (20 Rel. Vols), at 50° C., was added simultaneously, methanesulfonic acid (1 mol. eq.) and hydrogen peroxide (3 mol. eq.) over 1 hour. Following a further 1 hour, the mixture was quenched by the addition of sodium thiosulfate solution (1 M, 3 mol. eq.) and cooled to 20° C. Potassium hydroxide (49% (w/v), 7 mol eq.) was added and the layers were separated, retaining the lower aqueous layer. To this solution was added water (1.7 vols) and BuOAc (17 vols) and the pH was adjusted to 7 by the addition of hydrochloric acid (2.5 M). The layers were again separated, this time retaining the upper organic layer, which was washed with water (4.25 vols). The volume of the BuOAc solution was then reduced to approximately 8.5 Rel. Vols. by distillation under reduced pressure. Heptane (8.5 Rel. vols) was then added at approximately 80° C. and the mixture was cooled to 0° C. over 4 hours. The solid was filtered off, washed with a mixture of heptane and BuOAc (1.7 Rel. vols of each) then with heptane (3.4 vols) and then dried in vacuum oven at 50° C. Isolated yield of ZD6126 Phenol, from ZD6126 Alcohol was 85.1%; NMR and Mass spec characterisation data of ZD6126 Phenol was as described in Example 2.

### Example 4

### ZD6126 Alcohol (wherein R² are both methyl in formula (II) to ZD6126 Alkene of formula (III) wherein R² is methyl and R³ is hydrogen)

To a stirred mixture of ZD6126 Alcohol in THF (20 Rel. Vols), at 60° C., was added methanesulfonic acid (0.3 mol. eq.). The mixture was stirred for 9 hours, then quenched by the addition of sodium bicarbonate (0.35 mol. eq.). Water (6 vols) was added, followed by sodium chloride (solid) to cause phase separation. The upper organic layer was separated and washed with saturated brine, and the solvent was removed under reduced pressure, to provide ZD6126 Alkene as a solid. Isolated yield of ZD6126 Alkene, from ZD6126 Alcohol was approximately 84%: MS 382 [M+H]⁺ (75%), 323 [M−NHCOMe]⁺ (100%); δ^{H} ppm (500 MHz, DMSO-D₆) 1.91 (3 H, s, CCH₃), 2.05 (2 H, m, 2 x CH₂CH₂), 2.16 (3 H, s, COH₃), 2.18 (2 H, m, 2 x CH₂CH₂), 3.51 (3 H, s, OCH₃), 3.79 (3 H, s, OCH₃), 3.84 (3 H, s, OCH₃), 4.60 (1 H, ddd, J12.5, 3.5, 3.5, CH₂CH—NH), 5.14 (1 H, d, J1.5, ═CH₂), 5.48 (1 H, d, J1.5, ═CH₂), 6.79 (1 H, s, Ar—H), 7.31 (1H, d, J8, Ar—H), 7.43 (1H, dd, J8, 2, Ar—H), 7.52 (1H, d, J2, Ar—H), 8.45 (1H, d, J8.5, NH).

### Example 5

### ZD6126 Alcohol (wherein R² are both methyl in formula (II)) to ZD6126 Hydroperoxide of the formula (IV) wherein R² are both methyl)

To a slurry of ZD6126 Alcohol in BuOAc (20 Rel. Vols), at 30° C., under nitrogen, was added methanesulfonic acid in water (70% w/v, 1 mole equivalent) and 30% w/v hydrogen peroxide (4 mole equivalents) was added over 1 hour. After 2 hours, the mixture was cooled to 20° C. and the white solid filtered off. The solid was dissolved in a mixture of dichloromethane, methanol and hot ethyl acetate, then washed with saturated aqueous sodium bicarbonate solution, water and then saturated brine solution. The organic solution was evaporated to give ZD6126 Hydroperoxide as a white crystalline solid, in about 72% yield. [M+H]⁺: Found 416.2103 calculated for C₂₃H₂₉NO₆ 416.2073; δ^{H} ppm (500 MHz, DMSO-D₆) 1.5 (3 H, s, CHCH₃) 1.5 (s, 3 H, CHCH₃) 1.8 (1H, m) 1.9 (3 H, s, COCH₃) 2.0 (1 H, m) 2.1 (1 H, m) 2.5 (1 H, m) 3.5 (3 H, s, O CH₃) 3.8 (3 H, s, O CH₃ 3.8 (3 H, s, O CH₃) 4.6 (1 H, ddd, J11.5, 8, 8, CHN) 6.8 (1 H, s, Ar—H) 7.3 (1 H, d, J8, Ar—H) 7.3 (1 H, dd, J8, 2, Ar—H) 7.4 (1 Hd, J2, Ar—H) 8.4 (1 H, d, J8.5, Ar—H) 11.0 (1 H, s, OH); δ^{C} ppm (126 MHz, DMSO-D6 22.7, 26.4, 26.5, 30.2, 38.9, 48.2, 55.9, 60.6, 60.8, 82.2, 108.1, 120.1, 123.3, 124.3, 129.0, 132.5, 135.0, 139.6, 140.6, 144.3, 150.4, 152.5, 168.6.

### Example 6

### ZD6126 Alkene (wherein R² is methyl and R³ is hydrogen in formula (IV)) to ZD6126 Phenol

To a rapidly stirred solution of ZD6126 Alkene, in toluene (20 Rel. Vol.), at 50° C., was added simultaneously, methanesulfonic acid (1 mol. eq.) and hydrogen peroxide (3 mol. eq.) over 1 hour. Following a further 1 hour, the mixture was quenched by the addition of sodium thiosulfate solution (1 M, 3 mol. eq.) and cooled to 20° C. Potassium hydroxide (49% (w/v), 7 mol eq.) was added and the layers were separated, retaining the lower aqueous layer. To this solution was added water (1.7 vols) and BuOAc (17 vols) and the pH was adjusted to 7 by the addition of hydrochloric acid (2.5 M). The layers were again separated, this time retaining the upper organic layer, which was washed with water (4.25 vols). The volume of the BuOAc solution was then reduced to approximately 8.5 Rel. Vols. by distillation under reduced pressure. Heptane (8.5 Rel. vols) was then added at approximately 80° C. and the mixture was cooled to 0° C. over 4 hours. The solid was filtered off, washed with a mixture of heptane and BuOAc (1.7 Rel. vols of each) then with heptane (3.4 vols) and then dried in vacuum oven at 50° C. Yield of ZD6126 Phenol was 84%. Characterisation data for ZD6126 Phenol was as described in Example 2.

### Example 7

### ZD6126 Alcohol (wherein R² are both methyl in formula (II)) to ZD6126 Reactive Dimer of the formula (V) wherein R² are both methyl)

To a stirred solution of ZD6126 Alcohol in chlorobenzene (10 Rel. Vols), at 40° C., para-toluenesulfonic acid (0.40 equivalent. of a 70% (w/v) aq. Solution) and 50% (w/v) hydrogen peroxide (1.6 eq), were added over 30 minutes. The mixture was then quenched immediately by the addition of sodium thiosulfate solution (1 M, 3 mol. eq.). The organic solution contained ZD6126 Reactive Dimer in approximately 24% yield, as measured by HPLC. General method of isolation: The mixture is washed with potassium hydroxide (49%, 7 mol eq.), then water (1.7 vols). The remaining organic solution was then evaporated and ZD6126 Reactive Dimer was isolated from the residue by preparative HPLC. MS 797 [M+H]+ (100%), 382 (10%); δ^{C} ppm (126 MHz, DMSO-D6) 22.6, 26.2, 27.2, 30.1, 38.7, 48.1, 55.8, 60.4, 60.6, 81.6, 108.0, 120.2, 123.2, 124.2, 128.9, 132.6, 134.8, 139.5, 140.5, 143.9, 150.3, 152.4, 168.2; δ^{H} ppm (500 MHz, DMSO-D6) 1.5 (6 H, s, CHCH₃), 1.6 (6 H, s, CHCH₃), 1.80 (m, 2 H), 1.90 (6 H, s, COCH₃), 2.0 (2 H, m, CH₂CH₂), 2.2 (2 H, m, CH₂CH₂), 2.5 (2 H, m, CH₂CH₂), 3.5 (6 H, s, OCH₃), 3.8 (6 H, s, OCH₃), 3.8 (6 H, s, OCH₃), 4.6 (2 H, ddd, J12, 8.5, 7.5, CH₂CH—NH) 6.8 (2 H, s, Ar—H), 7.3 (2 H, d, J8, Ar—H), 7.3 (2 H, dd, J8, 2, Ar—H), 7.5 (2H, d, J2, Ar—H), 8.4 (2 H, d, J8.5, NH).

### Example 8

### ZD6126 Hydroperoxide of the formula (IV) wherein R² are both methyl) to ZD6126 Phenol

To a rapidly stirred solution of ZD6126 Hydroperoxide, in toluene (20 Rel. Vol.), at 50° C., was added methanesulfonic acid (2 mol. eq.) over 5 min. Following a further 1 hour, the mixture was quenched by the addition of sodium thiosulfate solution (2 M, 3 mol. eq.) and saturated sodium bicarbonate solution (2 Rel Vols.) and left to stir at ambient overnight. The solid was then filtered-off, washed with water (10 Rel Vols.) and toluene (10 Rel Vols.), then dried to give ZD6126 Phenol in 90% yield. Characterisation data for the ZD6126 Phenol was as described in Example 2 above.

### Example 9

### ZD6126 Reactive Dimer of the formula (V) wherein R² are both methyl) to ZD6126 Phenol

To a rapidly stirred solution of ZD6126 Reactive Dimer, in toluene (25 Rel. Vol.), at 50° C., was added simultaneously, methanesulfonic acid (2 mol. eq.) and hydrogen peroxide (6 mol. eq.) over 3 min. Following a further 2 hours, the mixture was neutralised by the addition of triethylamine, then diluted with ethanol (30 Vols). Conversion to ZD6126 Phenol was 82%, as measured by HPLC analysis. Characterisation data for the ZD6126 Phenol was as described in Example 2 above.

1. A process for the preparation of ZD6126 Phenol: 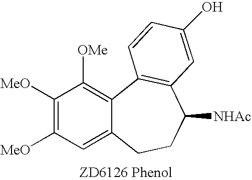
from a ZD6126 Alcohol of formula (II): 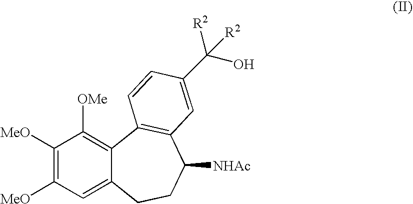
wherein R² are each independently hydrogen, C₁₋₄alkyl or aryl which comprises:
reacting said ZD6126 Alcohol of formula (II) with an acid catalyst and an oxidising agent.

2. A process according to claim 1, wherein the acid catalyst is an sulfonic acid.

3. A process according to claim 1, wherein the acid catalyst is methanesulfonic acid.

4. A process according to claim 1, wherein the reaction is carried out in the presence of a solvent selected from an aromatic solvent, an ester and an ether.

5. A process according to claim 1, wherein the reaction is carried out in an aromatic solvent selected from toluene and chlorobenzene, or a mixture of two or more of said solvents.

6. A process for the preparation of ZD6126 Phenol: 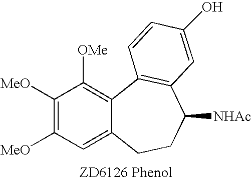
from an allocolchicine or an ester derivative thereof of formula (I): 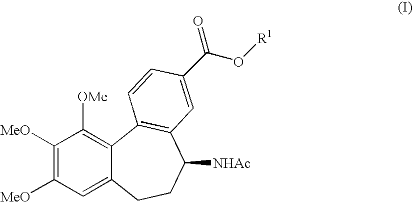
wherein R¹ is hydrogen, C₁₋₆alkyl or aryl; which comprises:
a) reacting said allocolchicine or an ester derivative thereof of formula (I) with a suitable organometallic reagent and/or a suitable reducing agent; in one or more ethereal solvents to form ZD6126 Alcohol of formula (II): 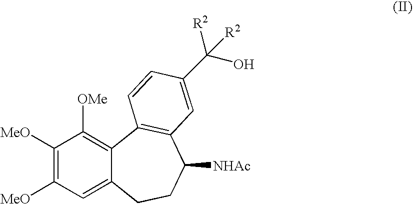
wherein R² is hydrogen, C₁₋₄alkyl or aryl; and
b) reacting ZD6126 Alcohol of formula (II) with an acid catalyst and an oxidising agent.

7. A process according to claim 6, wherein R¹ is C₁₋₄alkyl or aryl.

8. A process according to claim 6, wherein in step a) of the process the allocolchicine or an ester derivative thereof of formula (I) is reacted with a suitable organometallic reagent and wherein R¹ is C₁₋₄alkyl or aryl.

9. A process according to claim 6, wherein the organometallic reagent in step a) of the process is selected from a compound of the formula R²—X, wherein R² is as defined claim 6 and X is a magnesium halide or lithium.

10. A process according to claim 6, wherein the organometallic reagent in step a) is methyllithium.

11. A process according to claim 6, wherein the one or more etheral solvents is selected from tetrahydrofuran, diethyl ether, diethoxymethane, 2-ethoxyethylether, 2-methoxyethyl ether and dimethoxy ethane, or a mixture of one or more of said solvents.

12. A process according to claim 6, wherein in step a) the allocolchicine or an ester derivative thereof of formula (I) is added to a reaction mixture comprising the organometallic reagent.

13. A process according to claim 12, wherein the organometallic reagent is methyllithium.

14. A process according to claim 6, wherein the acid catalyst in step b) is a sulfonic acid.

15. A process according to claim 14, wherein the acid catalyst in step b) is methanesulfonic acid.

16. A process according to claim 6, wherein in step b) of the process is carried out in the presence of a solvent selected from an aromatic solvent, an ester and an ether.

17. A process according to claim 6, wherein in step b) of the process is carried out in the presence of an aromatic solvent selected from toluene and chlorobenzene, or a mixture of two or more of said solvents.

18. A process according to claim 6, wherein the process is effected in one stage, without isolation of ZD6126 Alcohol of formula (II).

19. A process according to claim 6, wherein R¹ is C₁₋₄alkyl.

20. A ZD6126 Alcohol of formula (II) 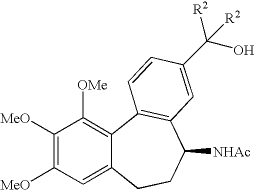
wherein R² are each independently hydrogen, C₁₋₄alkyl or aryl with the proviso that R² cannot both be methyl or both be hydrogen.

21. A process for the preparation of a ZD6126 Alcohol of the formula (II) 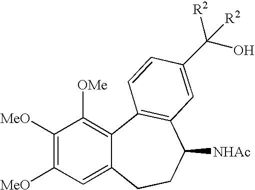
wherein R² are each independently hydrogen, C₁₋₄alkyl or aryl which comprises reacting allocolchicine or an ester derivative thereof the formula (I) as defined in claim 6 with a suitable organometallic reagent and/or suitable reducing agent in one or more ethereal solvents.

22. A method for preparing a ZD6126 phenol which comprises converting an optionally substituted methylol group of a ZD6126 alcohol into a hydroxy.

23. A ZD6126 Alkene of formula (III): 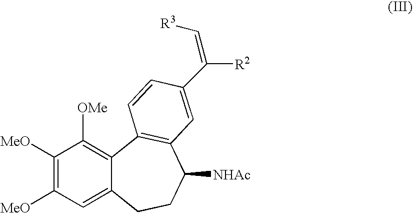
wherein R² is hydrogen, C₁₋₄alkyl or aryl and R³ is hydrogen or C₁₋₃alkyl.

24. A process for the preparation of ZD6126 Alkene of formula (III) as defined in claim 23 which comprises reacting a ZD6126 Alcohol of the formula (II) 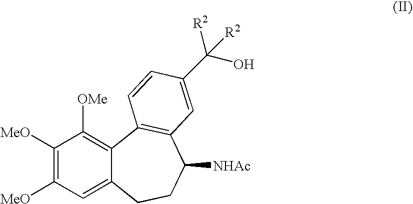
wherein R² are each independently hydrogen, C₁₋₄alkyl or aryl wherein at least one R² group is C₁₋₄alkyl, with an acid catalyst.

25. A process for the preparation of a ZD6126 Phenol which comprises reacting a ZD6126 Alkene of formula (III) as defined in claim 23 with an acid catalyst and an oxidising agent.

26. A ZD6126 Hydroperoxide of formula (IV): 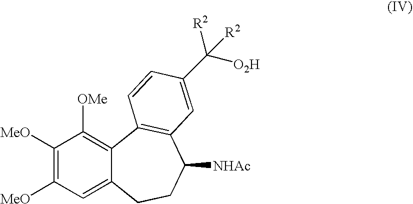
wherein R² are each independently hydrogen, C₁₋₄alkyl or aryl.

27. A process for the preparation of a ZD6126 Hydroperoxide of formula (IV) as defined in claim 26 which comprises reacting a ZD6126 Alcohol of the formula (II) 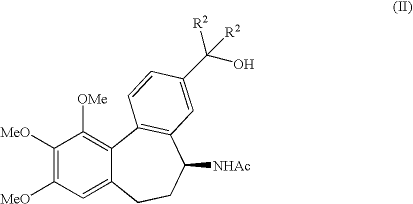
wherein R² are each independently hydrogen, C₁₋₄alkyl or with an acid catalyst and oxidising agent.

28. A process for the preparation of ZD6126 Phenol which comprises reacting a ZD6126 Hydroperoxide of formula (IV) as defined in claim 26 with an acid catalyst.

29. A ZD6126 Reactive Dimer of formula (V): 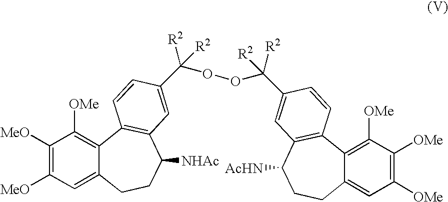
wherein R² are each independently hydrogen, C₁₋₄alkyl or aryl.
